(19) United States
(12) Patent Application Publication
(10) Pub. No.: US 2009/0012097 A1
(43) Pub. Date: Jan. 8, 2009

(54) Polycyclic 1,2,3,4-Tetrahydro-Isoquinoline Derivatives and Compositions Comprising Them As Ppar Modulators

(75) Inventors: Robert Epple, San Diego, CA (US); Christopher Cow, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/160,305

(22) PCT Filed: Jan. 25, 2007
(86) PCT No.: PCT/US07/02115

§ 371 Date: Aug. 11, 2008

Related U.S. Application Data
(60) Provisional application No. 60/763,623, filed on Jan. 30, 2006.

(51) Int. Cl. A61K 31/506; C07D 217/22; C07D 217/24; A61P 1/00; A61P 3/10; A61K 31/47

(52) U.S. Cl. 514/252.18; 546/141; 546/142; 514/309

(57) ABSTRACT

The invention provides compounds, pharmaceutical compositions comprising such compounds and methods of using such compounds to treat or prevent diseases or disorders associated with the activity of the Peroxisome Proliferator-Activated Receptor (PPAR) families.

### CROSS-REFERENCE TO RELATED APPLICATIONS

This patent application claims the benefit of priority under 35 U.S.C. §119(e) to U.S. Provisional Patent Application No. 60/763,623, filed Jan. 30, 2006. The disclosure of the priority application is incorporated herein by reference in its entirety and for all purposes.

### BACKGROUND OF THE INVENTION

1. Field of the Invention

The invention provides compounds; pharmaceutical compositions comprising such compounds and methods of using such compounds to treat or prevent diseases or disorders associated with the activity of the Peroxisome Proliferator-Activated Receptor (PPAR) families.

2. Background

Peroxisome Proliferator Activated Receptors (PPARs) are members of the nuclear hormone receptor super family, which are ligand-activated transcription factors regulating gene expression. Certain PPARs are associated with a number of disease states including dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, IBDs (irritable bowel disease), ulcerative colitis and Crohn's disease. Accordingly, molecules that modulate the activity of PPARs are useful as therapeutic agents in the treatment of such diseases.

### SUMMARY OF TH INVENTION

In one aspect, the present invention provides compounds of Formula I:

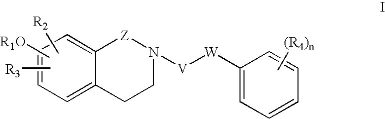

in which:

n is selected from 0, 1 and 2;

R₁ is —OCR₁₁R₁₂XCO₂R₁₃; wherein X is selected from a bond and C₁₋₄alkylene; and R₁₁ and R₁₂ are independently selected from hydrogen, C₁₋₄alkyl and C₁₋₄alkoxy; or R₁₁ and R₁₂ together with the carbon atom to which R₁₁ and R₁₂ are attached form C₃₋₁₂cycloalkyl; and R₁₃ is selected from hydrogen and C₁₋₆alkyl;

R₂ and R₃ are independently selected from hydrogen and C₁₋₆alkyl;

V is selected from a bond, C₁₋₄alkylene, —C(O)NR₈— and —X₁C(O)X₂—; wherein X₁ and X₂ are independently selected from a bond and C₁₋₄alkylene; R₈ is selected from hydrogen and C₁₋₆alkyl;

W is a divalent radical selected from (a) and (b):

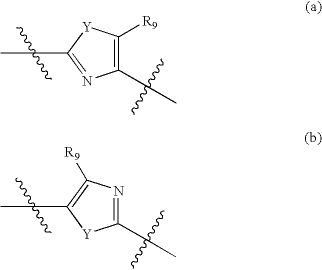

wherein Y is selected from O and S; and R₉ is selected from hydrogen and C₁₋₆alkyl;

Z is selected from —CH₂— and —C(O)—;

R₄ is selected from hydrogen, halo, C₁₋₆alkyl, halo-substituted-C₁₋₆alkyl, C₁₋₆alkoxy and halo-substituted-C₁₋₆alkoxy; and N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition that contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of PPAR activity can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which PPAR activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy; can be either straight-chained or branched. C₁₋₆alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group. “Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc. “C₆₋₁₀arylC₀₋₄alkyl” means an aryl as described above connected via a alkylene grouping. For example, “C₆₋₁₀arylC₀₋₄alkyl includes phenethyl, benzyl, etc.

Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, etc.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases in which modulation of one or more PPARs can prevent, inhibit or ameliorate the pathology and/or symptomology of the diseases, which method comprises administering to the animal a therapeutically effective amount of a compound of Formula I.

In one embodiment, with reference to compounds of Formula I: n is selected from 0 and 1; R₁ is —OCR₁₁R₁₂XCO₂R₁₃; wherein X is selected from a bond and C₁₋₄alkylene; and R₁₁ and R₁₂ are independently selected from hydrogen, C₁₋₄alkyl and C₁₋₄alkoxy; or R₁₁ and R₁₂ together with the carbon atom to which R₁₁ and R₁₂ are attached form C₃₋₁₂cycloalkyl; and R₁₃ is selected from hydrogen and C₁₋₄alkyl; R₂ and R₃ are independently selected from hydrogen and C₁₋₆alkyl; V is selected from a bond, C₁₋₄alkylene, —C(O)NR₈— and —X₁C(O)X₂—; wherein X₁ and X₂ are independently selected from a bond and C₁₋₄alkylene; R₈ is selected from hydrogen and C₁₋₆alkyl; W is a divalent radical selected from (a) an (b);

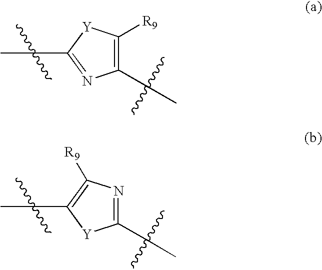

wherein Y is S; and R₉ is selected from hydrogen and C₁₋₆alkyl; Z is selected from —CH₂— and —C(O)—; and R₄ is selected from halo, C₁₋₆alkyl and halo-substituted-C₁₋₆alkyl.

In another embodiment, R₁ is selected from —CH₂CO₂H, —CH₂)₂CO₂H, —OC(CH₂)₂CO₂H and —OCH₂CO₂H; R₂ and R₃ are independently selected from hydrogen, methyl and methoxy; and R₄ is trifluoromethyl.

In another embodiment, V is selected from a bond, —C(O)—, —C(O)NH—, —C(O)N(CH₃)—, —CH₂— and —C(O)CH₂—.

Preferred compounds of the invention are selected from: 2-(2-(4-(4-(Trifluoromethyl)phenyl)-thiazol-2-ylcarbamoyl)-1,2,3,4-tetrahydroisoquinolin-6-yloxy)-2-methylpropanoic acid; 2-(2-N-(4-(4-(trifluoromethyl)phenyl)thiazol-2-yl)-N-methylcarbamoyl)-1,2,3,4-tetrahydroisoquinolin-6-yloxy)-2-methylpropanoic acid; 2-(2-(4-(4-(trifluoromethyl)phenyl)thiazol-2-yl)-1,2,3,4-tetrahydroisoquinolin-6-yloxy)-2-methylpropanoic acid; 2-(2-((4-(4-(trifluoromethyl)phenyl)thiazol-2-yl)(oxo)methyl)-1,2,3,4-tetrahydroisoquinolin-6-yloxy)-2-methylpropanoic acid; 2-(2-((4-(4-(trifluoromethyl)phenyl)thiazol-2-yl)methyl)-1,2,3,4-tetrahydroisoquinolin-6-yloxy)-2-methylpropanoic acid; 2-Methyl-2-{2-[4-(4-trifluoromethyl-phenyl)-thiazol-2-ylmethyl]-1,2,3,4-tetrahydro-isoquinolin-7-yloxy}-propionic acid; 2-Methyl-2-{2-[4-methyl-2-(4-trifluoromethyl-phenyl)-thiazol-5-ylmethyl]-1,2,3,4-tetrahydro-isoquinolin-7-yloxy}-propionic acid; 2-Methyl-2-{2-[4-ethyl-2-(4-trifluoromethyl-phenyl)-thiazol-5-ylmethyl]-1,2,3,4-tetrahydro-isoquinolin-6-yloxy}-propionic acid; 2-Methyl-2-{2-[5-methyl-2-(4-trifluoromethyl-phenyl)-thiazol-4-ylmethyl]-1,2,3,4-tetrahydro-isoquinolin-5-yloxy}-propionic acid; 2-Methyl-2-{2-[5-(4-trifluoromethyl-phenyl)-thiazol-2-ylmethyl]-1,2,3,4-tetrahydro-isoquinolin-6-yloxy}-propionic acid; 2-Methyl-2-{2-[4-methyl-2-(4-trifluoromethyl-phenyl)-thiazol-5-ylmethyl]-1-oxo-1,2,3,4-tetrahydro-isoquinolin-7-yloxy}-propionic acid; 2-Methyl-2-{2-[4-methyl-2-(4-trifluoromethyl-phenyl)-thiazol-5-ylmethyl]-1-oxo-1,2,3,4-tetrahydro-isoquinolin-6-yloxy}-propionic acid; 2-Methyl-2-{2-[4-(4-trifluoromethyl-phenyl)-thiazole-2-carbonyl]-1,2,3,4-tetrahydro-isoquinolin-7-yloxy}-propionic acid; 2-Methyl-2-(2-{2-[4-(4-trifluoromethyl-phenyl)-thiazol-2-yl]-acetyl}-1,2,3,4-tetrahydro-isoquinolin-6-yloxy)-propionic acid; 2-Methyl-2-(2-{methyl-[4-(4-trifluoromethyl-phenyl)-thiazol-2-yl]-carbamoyl}-1,2,3,4-tetrahydro-isoquinolin-7-yloxy)-propionic acid; 2-Methyl-2-{5-methyl-2-[4-4-trifluoromethyl-phenyl)-thiazol-2-ylcarbamoyl]-1,2,3,4-tetrahydro-isoquinolin-6-yloxy}-propionic acid; and 2-Methyl-2-(5-methyl-2-{methyl-[4-(4-trifluoromethyl-phenyl)-thiazol-2-yl]-carbamoyl}-1,2,3,4-tetrahydro-isoquinolin-6-yloxy)-propionic acid.

Further preferred compounds and intermediates of the invention are detailed in the Examples, infra.

### Pharmacology and Utility

Compounds of the invention modulate the activity of PPARs and, as such, are useful for treating diseases or disorders in which PPARs contributes to the pathology and/or symptomology of the disease. This invention further provides compounds of this invention for use in the preparation of medicaments for the treatment of diseases or disorders in which PPARs contributes to the pathology and/or symptomology of the disease.

Such compounds may therefore be employed for the treatment of prophylaxis, dyslipidemia, hyperlipidemia, hypercholesteremia, atherosclerosis, atherogenesis, hypertriglyceridemia, heart failure, hyper cholesteremia, myocardial infarction, vascular diseases, cardiovascular diseases, hypertension, obesity, cachexia, HIV wasting syndrome, inflammation, arthritis, cancer, Alzheimer's disease, anorexia, anorexia nervosa, bulimia, skin disorders, respiratory diseases, ophthalmic disorders, IBDs (irritable bowel disease), ulcerative colitis and Crohn's disease. Preferably for the treatment of prophylaxis, dyslipidemia, hyperlipidemia, hypercholesteremia, atherosclerosis, atherogenesis, hypertriglyceridemia, cardiovascular diseases, hypertension, obesity, inflammation, cancer, skin disorders, IBDs (irritable bowel disease), ulcerative colitis and Crohn's disease.

Compounds of the invention can also be employed to treat long term critical illness, increase muscle mass and/or muscle strength, increase lean body mass, maintain muscle strength and function in the elderly, enhance muscle endurance and muscle function, and reverse or prevent frailty in the elderly.

Further, the compounds of the present invention may be employed in mammals as hypoglycemic agents for the treatment and prevention of conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome X. Preferably type-1 and type-2 diabetes, Impaired Glucose Metabolism (IGM), Impaired Glucose Tolerance (IGT) and Impaired Fasting Glucose (IFG).

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of the invention or a pharmaceutically acceptable salt thereof. For any of the above uses, the required dosage will vary depending on the mode of administration, the particular condition to be treated and the effect desired. The present invention also concerns: i) a compound of the invention or a pharmaceutically acceptable salt thereof for use as a medicament; and ii) the use of a compound of the invention or a pharmaceutically acceptable salt thereof for the manufacture of a medicament for preventing or treating any of the diseases or disorders described above.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

This invention also concerns a pharmaceutical composition comprising a therapeutically effective amount of a compound as described herein in combination with one or more pharmaceutically acceptable carriers.

Compounds of the invention can be administered in therapeutically effective amounts in combination with one or more therapeutic agents (pharmaceutical combinations).

Thus, the present invention also relates to pharmaceutical combinations, such as a combined preparation or pharmaceutical composition (fixed combination), comprising: 1) a compound of the invention as defined above or a pharmaceutical acceptable salt thereof; and 2) at least one active ingredient selected from:
a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid described in the patent application WO 03/043985, as compound 19 of Example 4, a non-glitazone type PPARγ agonist e.g. GI-262570;
b) hypolipidemic agents such as 3-hydroxy-3-methyl-glutaryl coenzyme A (EMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;
d) anti-hypertensive agents, e.g., loop diuretics such as ethacrynic acid, furosemide and torsemide; diuretics such as thiazide derivatives, chlorithiazide, hydrochlorothiazide, amiloride; angiotensin converting enzyme (ACE) inibitors such as benazopril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;
e) a HDL increasing compound;
f) Cholesterol absorption modulator such as Zetia® and KT6-971;
g) Apo-A1 analogues and mimetics;
h) thrombin inhibitors such as Ximelagatran;
i) aldosterone inhibitors such as anastrazole, fadrazole, eplerenone;
j) Inhibitors of platelet aggregation such as aspirin, clopidogrel bisulfate;
k) estrogen, testosterone, a selective estrogen receptor modulator, a selective androgen receptor modulator;
l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib ({N-{5-[4-(4-methyl-piperazino-methyl)-benzoylamido]-2-methylphenyl}-4-(3-pyridyl)-2-pyrimidine-amine}) described in the European patent application EP-A-0 564 409 as example 21 or 4-Methyl-N-[3-(4-methyl-imidazol-1-yl)-5-trifluoromethylphenyl]-3-(4-pyridin-3-yl-pyrimidin-2-ylamino)-benzamide described in the patent application WO 04/005281 as example 92; and
m) an agent interacting with a 5-HT₃ receptor and/or an agent interacting with 5-HT₄ receptor such as tegaserod described in the U.S. Pat. No. 5,510,353 as example 13, tegaserod hydrogen maleate, cisapride, cilansetron; or, in each case a pharmaceutically acceptable salt thereof; and optionally a pharmaceutically acceptable carrier.

Most preferred combination partners are tegaserod, imatinib, vildagliptin, metformin, a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid, a sulfonylurea receptor ligand, aliskiren, valsartan, orlistat or a statin such as pitavastatin, simvastatin, fluvastatin or pravastatin.

Preferably the pharmaceutical combinations contains a therapeutically effective amount of a compound of the invention as defined above, in a combination with a therapeutically effective amount of another therapeutic agent as described above, e.g., each at an effective therapeutic dose as reported in the art. Combination partners (1) and (2) can be administered together, one after the other or separately in one combined unit dosage form or in two separate unit dosage forms. The unit dosage form may also be a fixed combination.

The structure of the active agents identified by generic or trade names may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

In another preferred aspect the invention concerns a pharmaceutical composition (fixed combination) comprising a therapeutically effective amount of a compound as described herein, in combination with a therapeutically effective amount of at least one active ingredient selected from the above described group a) to m), or, in each case a pharmaceutically acceptable salt thereof.

A pharmaceutical composition or combination as described herein for the manufacture of a medicament for the treatment of for the treatment of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.

Such therapeutic agents include estrogen, testosterone, a selective estrogen receptor modulator, a selective androgen receptor modulator, insulin, insulin derivatives and mimetics; insulin secretagogues such as the sulfonylureas, e.g., Glipizide and Amaryl; insulinotropic sulfonylurea receptor ligands, such as meglitinides, e.g., nateglinide and repaglinide; insulin sensitizers, such as protein tyrosine phosphatase-1B (PTP-1B) inhibitors, GSK3 (glycogen synthase kinase-3) inhibitors or RXR ligands; biguanides, such as metformin; alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors, e.g. isoleucin-thiazolidide; DPP728 and LAF237, hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin, fluindostatin and rivastatin, squalene synthase inhibitors or FXR (liver X receptor) and LXR (farnesoid X receptor) ligands, cholestyramine, fibrates, nicotinic acid and aspirin. A compound of the present invention may be administered either simultaneously, before or after the other active ingredient, either separately by the same or different route of administration or together in the same pharmaceutical formulation.

The invention also provides for pharmaceutical combinations, e.g. a kit, comprising: a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Son's; 1991.

Compounds of Formula 4 can be prepared by proceeding as in reaction scheme 1:

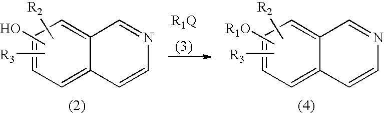

in which R₁, R₂ and R₃ are as defined for Formula I and Q is preferably chloro, bromo or iodo. Compounds of Formula 4 are prepared by reacting a compound of formula 2 with a compound of formula 3 in the presence of a suitable solvent (for example, DMF, and the like) and a suitable base (for example, potassium carbonate, and the like). The reaction is carried out in the temperature range of about 50 to about 150° C. and takes up to about 24 hours to complete.

Compounds of Formula 5 can be prepared by proceeding as in reaction scheme 2:

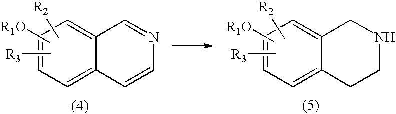

in which R₁, R₂ and R₃ are as defined for Formula I. Compounds of Formula 5 are prepared by reacting a compound of formula 4 with a suitable reducing agent (for example, hydrogen, and the like), a suitable solvent (for example, acetic acid, and the like) and a suitable catalyst (for example, platinum oxide, and the like). The reaction is carried out in a pressure range of about 40 to about 70 psi, a temperature range of about 0 to about 50° C. and takes up to about 24 hours to complete.

Compounds of Formula 8, wherein W is formula (a) as defined in the Summary of the Invention, can be prepared by proceeding as in reaction scheme 3:

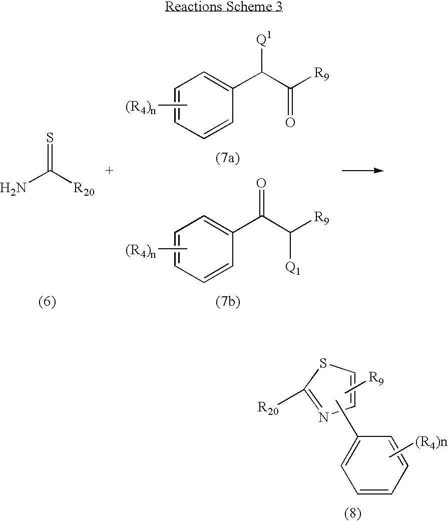

in which R₂₀ is selected from NH₂ and —COOR₂₃, —(CH₂)CN, and the like (R₂₃ is C₁₋₆alkyl); R₄, R₉ and n are as defined in the Summary of the Invention; and Q₁ is a halogen, preferably Cl, I or Br. Compounds of formula 8 are formed by reacting a compound of formula 6 with a compound of formula 7 in the presence of a suitable solvent (for example, acetone, ethanol, and the like). The reaction is carried out in the temperature range of about 50 to about 100° C. and takes up to about 6 hours to complete.

Compounds of Formula I, wherein V is methylene, can be prepared by proceeding as in reaction scheme 4:

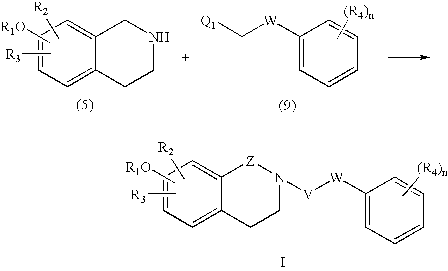

in which n, W, R₁, R₂, R₃ and R₄ are as defined for Formula I and Q₁ is chloro, bromo or iodo. Compounds of Formula I are prepared by reacting a compound of formula 5 with a compound of formula 9 in the presence of a suitable solvent (for example, 1,2-dichloroethane, and the like) and a suitable base (for example, diisopropylethylamine, and the like). The reaction is carried out in the temperature range of about 50 to about 120° C. and takes up to about 24 hours to complete.

Compounds of Formula I; wherein V is C(O), can be prepared by proceeding as in reaction scheme 5:

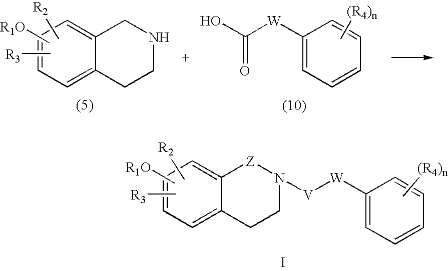

in which n, W, R₁, R₂, R₃ and R₄ are as defined for Formula I. Compounds of Formula I are prepared by reacting a compound of formula 5 with a compound of formula 10 in the presence of a suitable solvent (for example, THF, and the like), a suitable base (for example, diisopropylethylamine, and the like) and a suitable activator (for example, EDC/HOBt, and the like). The reaction is carried out in the temperature range of about 0 to about 50° C. and takes up to about 24 hours to complete.

Compounds of Formula I, wherein V is a bond, can be prepared by proceeding as in reaction scheme 6:

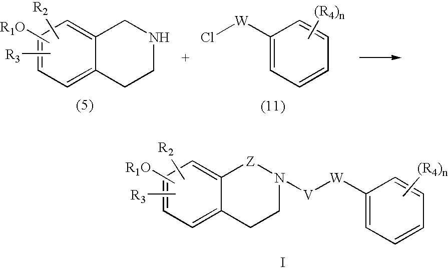

in which n, W, R₁, R₂, R₃ and R₄ are as defined for Formula I. Compounds of Formula I are prepared by reacting a compound of formula 5 with a compound of formula 11 in the presence of a suitable solvent (for example, dioxane, and the like), a suitable catalyst (for example, Pd₂(dba)₃, and the like), a suitable ligand (for example, phosphine ligands such as (tBU)₃PHBF₃, and the like), a suitable inorganic base (for example, Cesium carbonate, and the like) under a suitable-protective atmosphere (for example, argon, and the like). The reaction is carried out in the temperature range of about 80 to about 150° C. and takes up to about 24 hours to complete.

Compounds of Formula I, wherein V is a —C(O)NH—, can be prepared by proceeding as in reaction scheme 7:

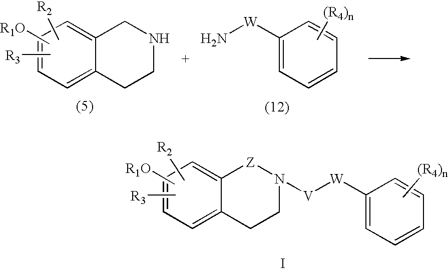

in which n, W, R₁, R₂, R₃ and R₄ are as defined for Formula I. Compounds of Formula I are prepared by reacting a compound of formula 5 with a compound of formula 12 in the presence of a suitable solvent (for example, THF, and the like), a suitable reagent (for example, triphosgene, CDI, and the like) and a suitable base (for example, triethylamine, and the like). The reaction is carried out in the temperature range of about 0 to about 50° C. and takes up to about 6 hours to complete.

Compounds of Formula I, where R₁ is CR₁₁R₁₂XCO₂R₁₃ (and R₁₃ is C₁₋₆alkyl), are converted to their corresponding acids (where R₁₃ is hydrogen) via a saponification reaction. The reacting proceeds in the presence of a suitable base (e.g., lithium hydroxide, or the like) and a suitable solvent mixture (e.g., THF/water, or the like) and is carried out in the temperature range of about 0° C. to about 50° C., taking up to about 30 hours to complete.

Detailed reaction conditions are described in the examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A cm pound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers hive distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme above; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following intermediates and examples that illustrate the preparation of compounds of Formula I according to the invention.

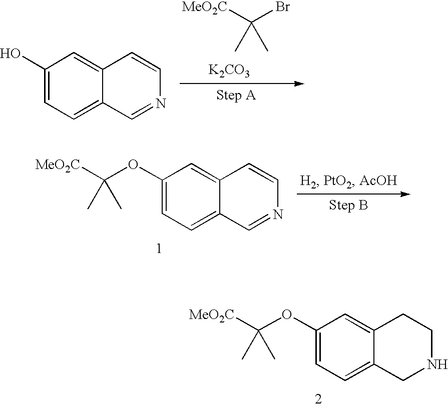

### Intermediate 2: Methyl 2-(1,2,3,4-tetrahydroisoquinolin-6-yloxy)-2-methylpropanoate

Step A: 6-Hydroxyisoquinoline (1.0 g, 6.9 mmol) and methyl 2-bromoisobutyrate (3.4 mL, 27.5 mmol) are dissolved in dry DMF (20 mL). Powdered potassium carbonate (3.8 g, 27.5 mmol) is added and the mixture is heated at 100° C. for 16 h. The mixture is cooled, diluted-with-ethyl acetate (40 mL), washed with water (3×50 mL) and brine (50 mL). The organic layer is dried (MgSO₄), filtered, evaporated and purified by silica gel chromatography (0-100% gradient, ethyl acetate in hexanes) to provide methyl 2-(isoquinolin-6-yloxy)-2-methylpropanoate 1 (1.25 g, 74%) as a colorless oil. MS calcd. for C₁₄H₁₆NO₃ (M+H⁺) 246.1, found 246.1.

Step B: Methyl 2-(isoquinolin-6-yloxy)-2-methylpropanoate 1 (1.1 g, 4.5 mmol) is dissolved in glacial acetic acid (20 mL). PtO₂ (˜50 mg, cat) is added, then the vessel is pressurized with H₂ to 55 psi and evacuated 3 times, then pressurized to 55 psi and shaken at r.t. for 16 h. The mixture is filtered through celite, then evaporated to dryness, dissolved in dichloromethane and evaporated to give the title compound 2 (0.99 g, 89%) as a yellow oil. TH-NMR (400 MHz, CDCl₃) δ=6.96 (d, J=8.4 Hz, 1H), 6.70 (dd, J=2.4, 8.4 Hz, 1H), 6.64 (d, J=2.4 Hz, 1H), 4.243 (s, 2H), 3.76 (s, 3H), 3.40 (s, 2H), 3.04 (t, J=6.4 Hz, 2H), 1.59 (s, 6H). MS calcd. for C₁₄H₂₀NO₃ (M+H⁺) 250.1, found 250.1.

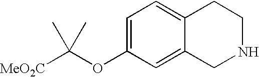

### Intermediate 3: Methyl 2-(1,2,3,4-tetrahydroisoquinolin-7-yloxy)-2-methylpropanoate

Following the procedure for Intermediate 2, except substituting 7-hydroxyisoquinoline for 6-hydroxyisoquinoline, the title compound is prepared as a yellow oil: ¹H-NMR (400 MHz, CDCl₃) δ=7.04 (d, J=8.4 Hz, 1H), 6.74 (dd, J=2.4, 8.4 Hz, 1H), 6.52 (d, J=2.4 Hz, 1H), 4.23 (s, 2H), 3.76 (s, 3H), 3.43 (d, J=5.2 Hz, 2H), 3.02 (t, J=6.0 Hz, 2H), 1.55 (s, 6H). MS calcd. for C₁₄H₂₀NO₃ (M+H⁺) 250.1, found 250.1.

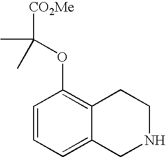

### Intermediate 4: Methyl 2-(1,2,3,4-tetrahydroisoquinolin-5-yloxy)-2-methylpropanoate

Following the procedure for Intermediate 2, except substituting 5-hydroxyisoquinoline for 6-hydroxyisoquinoline, the title compound is prepared as a yellow oil: ¹H-NMR (400 MHz, CDCl₃) δ=7.07 (t, J=8.0 Hz, 1H), 6.70 (d, J=7.6 Hz, 1H), 6.49 (d, J=8.0 Hz, 1H), 4.21 (s, 2H), 3.74 (s, 3H), 3.36 (t, J=6.4 Hz, 2H), 2.96 (t, J=6.4 Hz, 2H), 1.60 (s, 6H). MS calcd. for C₁₄H₂₀NO₃ (M+H⁺) 250.1, found 250.2.

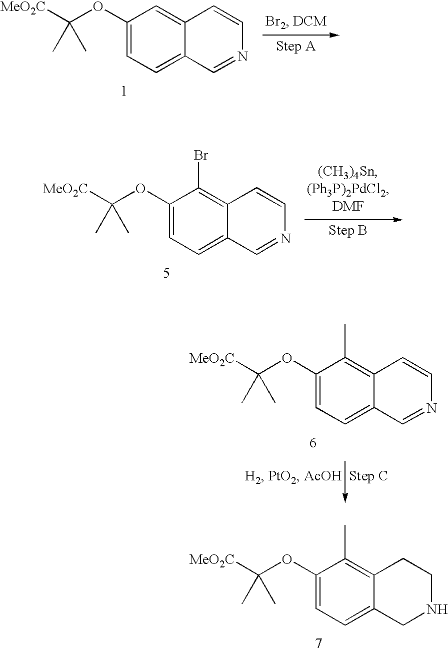

### Intermediate 7: Methyl 2-(1,2,3,4-tetrahydro-5-methylisoquinolin-6-yloxy)-2-methylpropanoate

Step A: Methyl 2-(isoquinolin-6-yloxy)-2-methylpropanoate 1 (55 mg, 0.22 mmol) is dissolved in dichloromethane (4 mL). Bromine (18 μL, 0.34 mmol) is added and stirred at r.t for 2 h. The solvent is removed by evaporation, providing methyl 2-(5-bromoisoquinolin-6-yloxy)-2-methylpropanoate 5 as a yellow oil. ¹H-NMR (400 MHz, CDCl₃) δ=9.53 (s, 1H), 8.45 (d, J=6.8 Hz, 1H), 8.35 (d, J=6.4 Hz, 1H), 8.12 (d, J=9.2 Hz, 1H), 7.27 (d, J=9.2 Hz, 1H), 3.75 (s, 3H), 1.73 (s, 6H). MS calcd. for C₁₄H₁₅BrNO₃ (M+H⁺) 324.0, found 324.0.

Step B: Methyl 2-(5-bromoisoquinolin-6-yloxy)-2-methylpropanoate 5 (500 mg, 1.5 mmol) is dissolved in DMF (0.5 mL). (CH₃)₄Sn (280 μL, 2.0 mmol) and (Ph₃P)₂PdCl₂ (220 mg, 0.31 mmol) are added and the mixture is subjected to microwave (180° C.) for 10 min in a sealed tube. The mixture is diluted with water (5 mL), extracted into EtOAc (10 mL) and washed with water (2×5 mL) and brine (5 mL). The organic layer is dried (MgSO₄), filtered, concentrated, and purified on reverse phase HPLC (H₂O/MeCN gradient) to afford methyl 2-(5-methylisoquinolin-6-yloxy)-2-methylpropanoate 6 (310 mg, 76%) as a colorless oil: ¹H-NMR (400 MHz, CDCl₃) δ=9.18 (br. s, 1H), 8.5 (br. s, 1H), 7.84 (d, J=9.2 Hz, 1H), 7.75 (s, 1H), 7.04 (d, J=8.8 Hz, 1H), 3.74 (s, 3H), 2.41 (s, 3H), 1.59 (s, 6H). MS calcd. for C₁₅H₁₈NO₃ (M+H⁺) 260.1, found 260.1.

Step C: Methyl 2-(5-methylisoquinolin-6-yloxy)-2-methylpropanoate 6 (310 mg, 1.2 mmol) is dissolved in glacial acetic acid (20 mL). PtO₂ (˜20 mg, cat) is added, then the vessel is pressurized with H₂ to 55 psi and evacuated 3 times, then pressurized to 55 psi and shaken at r.t. for 16 h. The mixture is filtered through celite, made basic by addition of 1.0 N NaOH, and extracted with ethyl acetate (40 mL). Washed with brine (20 mL), dried (MgSO₄), filtered and evaporated to give the title compound 7 (0.28 g, 91%) as a yellow oil. ¹H-NMR (400 MHz, CDCl₃) δ=6.81 (d, J=8.4 Hz, 1H), 6.57 (d, J=8.4 Hz, 1H), 4.26 (s, 2H), 3.77 (s, 3H), 3.46 (s, 2H), 3.00 (m, 2H), 2.13 (s; 3H), 1.59 (s, 6H). MS calcd. for C₁₅H₂₂NO₃ (M+H⁺) 264.2, found 264.1.

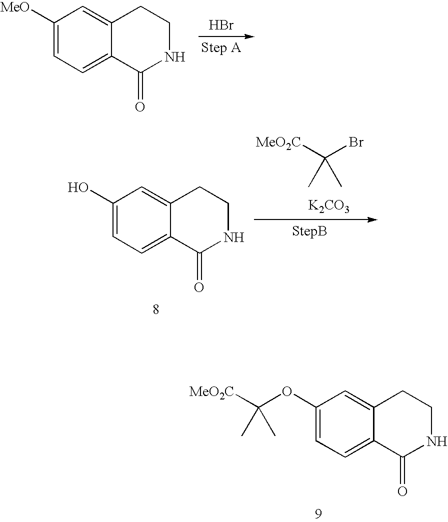

### Intermediate 9: Methyl 2-(1,2,3,4-tetrahydro-1-oxoisoquinolin-6-yloxy)-2-methylpropanoate

Step A: 3,4-dihydro-6-methoxyisoquinolin-1-(2H)-one (760 mg, 4.3 mmol) is dissolved in 48% aqueous HBr (5 mL) and heated at 100° C. for 72 h. The mixture is cooled and poured into a saturated solution of NaHCO₃ (50 mL) and extracted with ethyl acetate (2×20 mL). The organic fractions are combined, washed with brine (20 mL), dried (MgSO₄), filtered and evaporated to give crude 3,4-dihydro-6-hydroxyisoquinolin-1(2H)-one 8 (193 mg, 27%), which is used in Step B without further purification.

Step B: 3,4-dihydro-6-hydroxyisoquinolin-1(2H)-one 8 (193 mg, 1.2 mmol) and methyl 2-bromoisobutyrate (0.60 mL, 4.7 mmol) is dissolved in anhydrous DMF (10 mL). Powdered potassium carbonate (0.65 g, 4.7 mmol) is added and the mixture is heated at 100° C. for 16 h. The mixture is cooled, diluted with ethyl acetate (20 mL), washed with water (3×20 mL) and brine (20 mL). The organic layer is dried (MgSO₄), filtered, and evaporated to provide the title compound 9. MS calcd. for C₁₄H₁₈NO₄ (M+H⁺) 264.1, found 264.1.

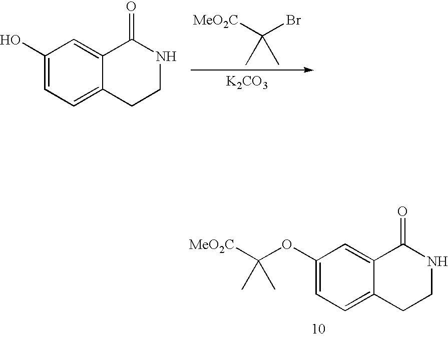

### Intermediate 10: Methyl 2-(1,2,3,4-tetrahydro-1-oxoisoquinolin-7-yloxy)-2-methylpropanoate

Following the procedure for Intermediate 10, Step B, except substituting 3,4-dihydro-7-hydroxyisoquinolin-1(2H)-one for 3,4-dihydro-6-hydroxyisoquinolin-1(2H)-one, the title compound is prepared as a yellow oil. MS calcd. for C₁₄H₁₈NO₄ (M+H⁺) 264.1, found 264.1.

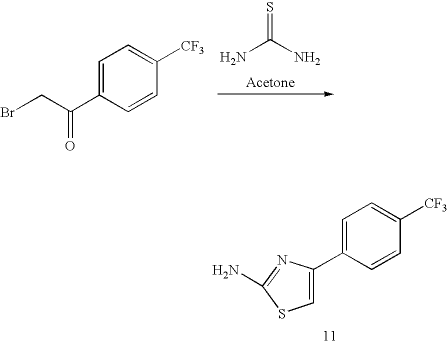

### Intermediate 11: 4-(4-Trifluoromethyl-phenyl)-thiazol-2-ylamine

2-Bromo-1-(4-trifluoromethyl-phenyl)-ethanone (10 g, 37.4 mmol) and thiourea (2.85 g, 37.4 mmol) are dissolved in dry acetone (100 mL) and heated at reflux for 2 h. The solution is cooled and stirred at rt for 2 h, then filtered and washed with acetone to give 4-(4-trifluoromethyl-phenyl)-thiazol-2-ylamine 11 (9.35 g, 100%) as white crystals. ¹H-NMR (400 MHz, DMSO-d₆) δ=8.30 (br. s, 2H), 7.98 (d, J=8.0 Hz, 2H), 7.84 (d, J=8.0 Hz, 2H), 7.42 (s, 1H). MS calcd. for C₁₀H₈F₃N₂S (M+H⁺) 245.0, found 245.1.

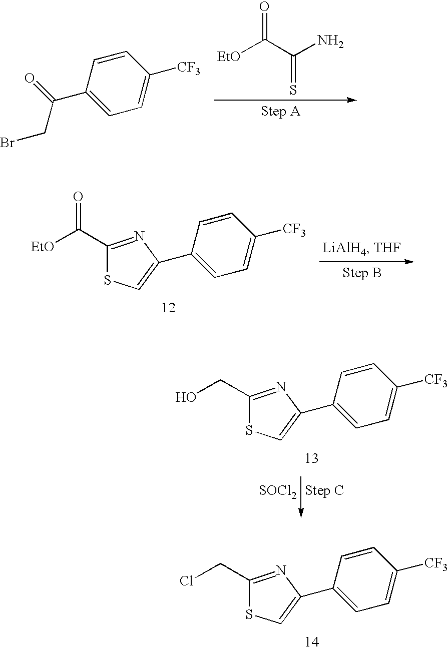

### Intermediate 14: 2-(Chloromethyl)-4-(4-(trifluoromethyl)phenyl)thiazole

Step A: 2-bromo-1-(4-(trifluoromethyl)phenyl)ethanone (17 g, 63 mmol) and ethyl thiooxamate (8.4 g, 63 mmol) are heated at reflux in EtOH (20 mL) for 2 h. The mixture is cooled, filtered and washed with MeOH to provide ethyl 4-(4-(trifluoromethyl)phenyl)thiazole-2-carboxylate 12 (13.3 g, 70%) as a white powder: ¹H-NMR (400 MHz, CDCl₃) δ=8.07 (d, J=8.0 Hz, 2H), 7.85 (s, 1H), 7.69 (d, J=8.4 Hz, 2H), 4.51 (q, J=7.2 Hz, 2H), 1.46 (t, J=7.2 Hz, 3H). MS calcd. for C₁₃H₁₁F₃NO₂S (M+H⁺) 302.0, found 302.0.

Step B: Ethyl 4-(4-(trifluoromethyl)phenyl)thiazole-2-carboxylate 12 (5.0 g, 16.6 mmol) is dissolved in dry THF (35 mL) and cooled to 0° C. LiAlH₄ (25 mL of 1.0 N in THF) is added dropwise to the solution and stirred at 0° C. for 1 h. The reaction is quenched with dropwise addition of 1 N HCl (50 mL), and extracted into ethyl acetate (50 mL). The organic layer is washed with brine (20 mL); dried (MgSO₄), filtered, and evaporated to give (4-(4-(trifluoromethyl)phenyl)thiazol-2-yl)methanol 13 (3.7 g, 78%) as a yellow oil. MS calcd. for C₁₁H₉F₃NOS (M+H⁺) 260.0, found 260.0.

Step C: (4-(4-(trifluoromethyl)phenyl)thiazol-2-yl)methanol 13 (1.68 g, 6.5 mmol) is dissolved in dry THF (20 mL). Thionyl chloride (0.94 mL, 13.0 mmol) is added and the mixture is stirred at r.t. for 2 h, then is poured into a saturated solution of NaHCO₃ (40 mL) and extracted with ethyl acetate (40 mL). The organic layer is dried (MgSO₄), filtered and evaporated to give the title compound (1.47 g, 82%) as a yellow solid: ¹H-NMR (400 MHz, CDCl₃) δ=8.00 (d, J=8.0 Hz, 2H), 7.68 (d, J=8.0 Hz, 2H), 7.63 (s, 1H), 4.92 (s, 2H). MS calcd. for C₁₁H₈ClF₃NS (M+H⁺) 278.0, found 278.1.

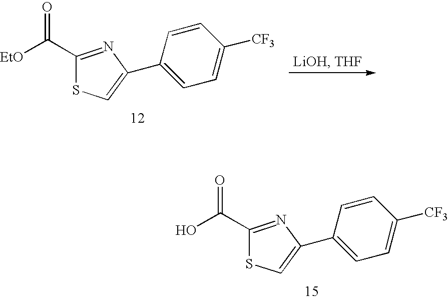

### Intermediate 15: 4-(4-(Trifluoromethyl)phenyl)thiazole-2-carboxylic acid

Ethyl 4-(4-(trifluoromethyl)phenyl)thiazole-2-carboxylate 12 (4.5 g, 15 mmol) is dissolved in THF (100 mL), then 1 N LiOH (22 mL, 22 mmol) is added and the mixture is heated at reflux for 2 h. The reaction is acidified with 1 N HCl (100 mL) and extracted with ethyl acetate (2×200 mL). The organic layers are combined, washed with brine (50 mL), dried (MgSO₄), filtered, and evaporated. The residue is recrystallized from ethyl acetate/hexane and filtered to give the title compound 15 (2.28 g, 56%) as a white solid: ¹H-NMR (400 MHz, CDCl₃) δ=8.05 (d, J=8.4 Hz, 2H), 7.98 (s, 1H), 7.73 (d, J=8.0 Hz, 21). MS calcd. for C₁₁H₇F₃NO₂S (M+H⁺) 274.0, found 274.0.

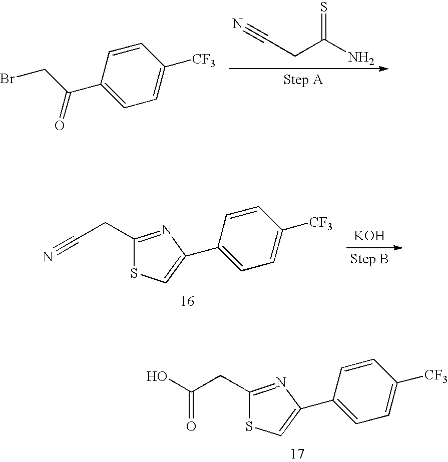

### Intermediate 17: 2-(4-(4-(trifluoromethyl)phenyl)thiazol-2-yl)acetic acid

Step A: 2-bromo-1-(4-(trifluoromethyl)phenyl)ethanone (5.3 g, 20 mmol) and 2-cyanothioacetamide (2.0 g, 20 mmol) are heated at reflux in EtOH (20 mL) for 2 h. The solvent is evaporated to provide crude 2-(4-(4-(trifluoromethyl)phenyl)thiazol-2-yl)acetonitrile 16 which is used in Step B without further purification. MS calcd. for C12H8F3N2S (M+H+) 269.0, found 269.0.

Step B: 2-(4-(4-(trifluoromethyl)phenyl)thiazol-2-yl)acetonitrile 16 (20 mmol) is dissolved in a mixture of methoxyethanol (100 mL) and water (20 mL). Potassium hydroxide (6.7 g, 120 mmol) is added and the mixture is heated at 100° C. for 16 h. The mixture is acidified with 1 N HCl to pH 1 and extracted with ethyl acetate (2×200 mL). The organic layers are combined, washed with water (200 mL) and brine (50 mL), dried (MgSO₄), filtered and evaporated to give the title compound 17 (4.3 g, 76%) as a white solid; 1H-NMR (400 MHz, CDCl3) δ=7.97 (d, J=8.4 Hz, 2H), 7.69 (d, J=8.4 Hz, 2H), 7.60 (s, 1H), 4.21 (s, 2H). MS calcd. for C12H9F3NO2S (M+H+) 288.0, found 288.0.

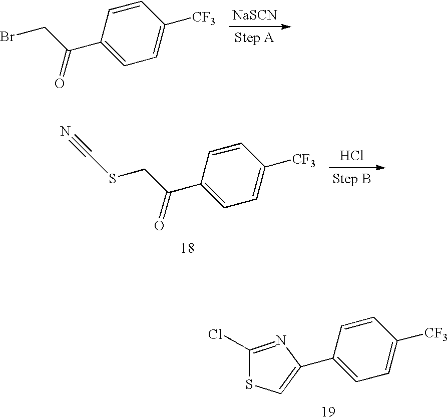

### Intermediate 19: 2-chloro-4-(4-(trifluoromethyl)phenyl)thiazole

Step A: 2-bromo-1-(4-(trifluoromethyl)phenyl)ethanone (2.0 g, 7.5 mmol) and NaSCN (728 mg, 9.0 mmol) are heated at reflux in EtOH (10 mL) for 2 h. The mixture is acidified with 1 N HCl (10 mL), extracted with ethyl acetate (20 mL), washed with brine (10 mL), dried (MgSO4), filtered and evaporated to provide crude 1-(4-(trifluoromethyl)phenyl)-2-thiocyanatoethanone 18 which is used in Step B without further purification. MS calcd. for C10H7F3NOS (M+H+) 246.0, found 246.0.

Step B: 1-(4-(trifluoromethyl)phenyl)-2-thiocyanatoethanone 18 (7.4 mmol) is dissolved in a mixture of THF (5 mL) and 4.0 N HCl in dioxane (5 mL) and heated at reflux for 16 h. The mixture is cooled and neutralized with saturated NaHCO3 solution, then extracted with EtOAc (20 mL), dried (MgSO4), filtered, evaporated and purified on reverse phase HPLC (H2O/MeCN gradient) to afford the title compound 19 (360 mg, 18%): 1H-NMR (400 MHz, CDCl3) δ=7.97 (d, J=8.4 Hz, 2H), 7.67 (d, J=8.4 Hz, 2H), 7.47 (s, 1H). MS calcd. for C10H6ClF3NS (M+H+) 264.0, found 264.0.

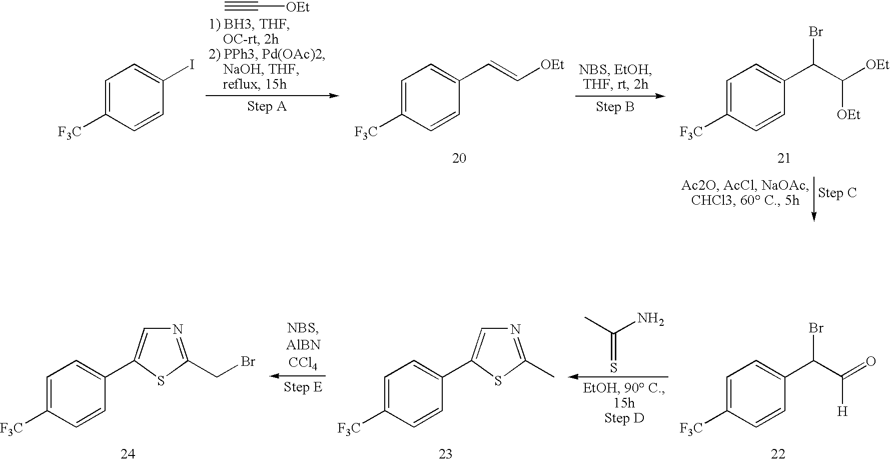

### Intermediate 24: 2-(bromomethyl)-5-(4-(trifluoromethyl)phenyl)thiazole

Step A: To the solution of ethyl ethynyl ether (6.0 g, 85.6 mmol) in THF (100 mL) is added borane-tetrahydrofuran complex (1.0 mol in THF, 28.53 mL, 28.53 mmol) at 0° C., then the mixture is warmed to RT and stirred two hours. The above solution is added to the mixture of 1-Iodo-4-trifluoromethyl-benzene (19.0 g, 71.33 mmol), triphenylphosphine (598 mg, 2.28 mmol), palladium(II) acetate (128 mg, 0.571 mmol) and sodium hydroxide (8.5 g, 214.0 mmol) in THF (200 mL). The above mixture is heated at reflux for 15 hours, then cooled, diluted with EtOAc (1000 mL), washed with saturated Na₂CO₃ and brine and water. The organic layer is dried (MgSO₄), filtered and concentrated to give crude product, which is purified by silic gel chromatography with ether/hexane to give 1-(2-ethoxy-vinyl)-4-trifluoromethyl-benzene 20 (9.40 g, 60.0%) as a white solid: MS calcd. for C₁₁H₁₂F₃₀ (M+H⁺) 217.1, found 217.1.

Step B: 1-(2-ethoxy-vinyl)-4-trifluoromethyl-benzene 20 (9.24 g, 42.74 mmol) is dissolved in the solution of EtOH (200 mL), then NBS (7.61 g, 42.74 mmol) is added and stirred at rt for 2 hs. The mixture is concentrated to give crude product, which is purified by silic gel chromatography by EtOAc/hexane to give 1-(1-bromo-2,2-diethoxyethyl)-4-trifluoromethyl-benzene 21 (12.87 g, 88.2%) as a colorless oil.

Step C: 1-(1-Bromo-2,2-diethoxy-ethyl)-4-trifluoromethyl-benzene 21 (10.57 g, 30.78 mmol) is dissolved in the solution of chloroform (180 mL), then Ac₂O (2.90 mL, 30.78 mmol), NaOAc.3H₂O (2.52 g, 18.5 mmol) and AcCl (1.53 mL, 21.6 mmol) are added and stirred at 56° C. for 5 hs. The mixture is diluted with chloroform (140 mL) and washed with saturated NaHCO₃ and brine. The organic layer is dried (MgSO₄), filtered and concentrated to give crude biphenyl-4-yl-bromo-acetaldehyde 22 as a thick oil (8.20 g, 100%), which is used for next reaction without purification.

Step D: The aldehyde 22 (100 mg, 0.375 mmol) is dissolved in EtOH (1.0 mL), then thioacetamide (28.13 mg, 0.375 mmol) is added and the mixture is stirred at 90° C. for 15 hs. The solution is diluted EtOAc (50 mL) and washed with saturated NaHCO₃ (30 mL) and brine (10 mL). The organic layer is dried (MgSO₄), filtered and concentrated to give crude product, which is purified by silic gel chromatography by EtOAc/hexane to give 2-methyl-5-(4-trifluoromethyl-phenyl)-thiazole 23 (43 mg, 46.8%) as a white solid: ¹H-NMR (400 MHz, CDCl₃) δ=7.78 (s, 1H), 7.51-7.56 (m, 5H), 4.28-7.41 (m, 4H), 2.69 (s, 3H). MS calcd. for C₁₆H₁₄NS (M+H⁺) 252.1, found 252.0.

Step E: 2-methyl-5-(4-trifluoromethyl-phenyl)-thiazole 23 (1.3 g, 5.3 mmol) and N-bromosuccinimide (1.24 g, 6.9 mmol) are suspended in carbon tetrachloride (100 mL) and heated to 40° C. 2,2-azo-bis-isobutyronitrile (AIBN, 88 mg, 0.53 mmol) is added and the mixture is heated to 70° C. for 12 h. The reaction mixture is cooled, diluted with water (100 mL), extracted into dichloromethane (40 mL), dried (MgSO₄), filtered, evaporated and purified on silica gel (EtOAc/Hexane gradient) to provide the title compound 24 as a yellow powder (935 mg, 54%). MS calcd. for C₁₁H₈BrF₃NS (M+H⁺) 321.9, found 252.00.

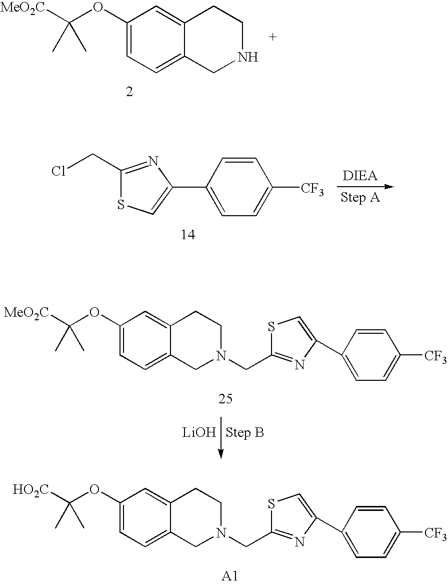

### Example A1

### 2-(2-((4-(4-(trifluoromethyl)phenyl)thiazol-2-yl)methyl)-1,2,3,4-tetrahydroisoquinolin-6-yloxy)-2-methylpropanoic acid

Step A: The amine 2 (40 mg, 0.16 mmol), the chloride 14 (45 mg, 0.16 mmol) and diisopropylethylamine (79 μL, 0.48 mmol) are dissolved in 1,2-dichloroethane (3 mL) and heated to 80° C. for 16 h. The solvent is removed in vacuo to give crude methyl 2-(2-((4-(4-(trifluoromethyl)phenyl)thiazol-2-yl)methyl)-1,2,3,4-tetrahydroisoquinolin-6-yloxy)-2-methylpropanoate 25, which is used in Step B without further purification. MS calculated for C₂₅H₂₆F₃N₂O₃S (M+H⁺) 491.2, found 491.1.

Step B: The residue from Step A is dissolved in THF (3 mL) and 1 N LiOH (1 mL). The mixture is stirred at 70° C. for 12 h then acidified with 1 N HCl (˜5 mL) and extracted with EtOAc (10 mL), dried (MgSO₄), filtered, evaporated and purified on reverse phase HPLC (H₂O/MeCN gradient) to afford the title compound A1 (29 mg, 38%): ¹H-NMR (400 MHz, CDCl₃) δ=7.99 (d, J=8.0 Hz, 2H), 7.74 (s, 1H), 7.70 (d, J=8.0 Hz, 2H), 6.93 (m, 1H), 6.74 (m, 2H), 4.74 (s, 2H), 4.47 (s, 2H), 3.55 (br s, 2H), 3.11 (br s, 2H), 1.60 (s, 6H). MS calculated for C₂₄H₂₄F₃N₂O₃S (M+H⁺) 477.2, found 477.1.

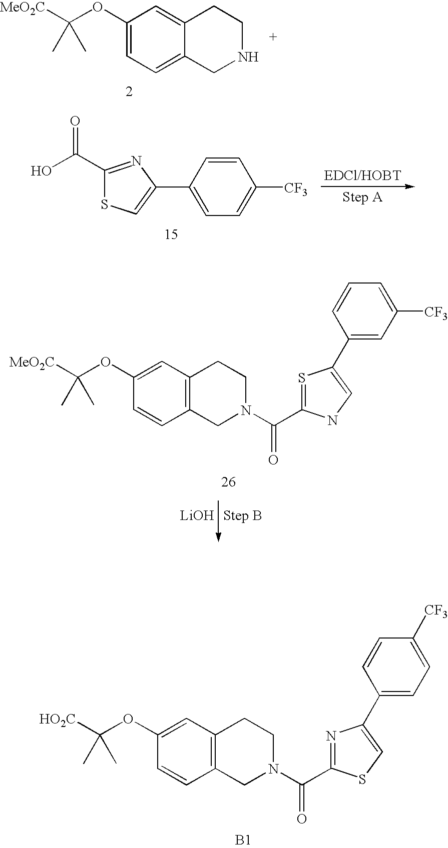

### Example B1

### 2-(2-((4-(4-(trifluoromethyl)phenyl)thiazol-2-yl)(oxo)methyl)-1,2,3,4-tetrahydroisoquinolin-6-yloxyy-2-methylpropanoic acid

Step A: 4-(4-(Trifluoromethyl)phenyl)thiazole-2-carboxylic acid 15 (55 mg, 0.20 mmol) is dissolved in THF (4 mL) and cooled to 0° C. Diisopropylethylamine (73 μL, 0.44 mmol) and 1-[3-(dimethylamino)propyl]-3-ethylcarbodiimide (EDCI, 42 mg, 0.22 mmol) are added and stirred at 0° C. for 5 min, then 1-hydroxybenzotriazole hydrate (HOBT, 34 mg, 0.22 mmol) is added and stirred a further 30 min. Methyl 2-(1,2,3,4-tetrahydroisoquinolin-6-yloxy)-2-methylpropanoate 2 (50 mg, 0.20 mmol) is added and the mixture is stirred at rt for 14 h. The solvent is removed in vacuo to provide crude methyl 2-(2-((4-(4-(trifluoromethyl)phenyl)thiazol-2-yl)(oxo)methyl)-1,2,3,4-tetrahydroisoquinolin-6-yloxy)-2-methylpropanoate 26, which is used in Step B without further purification. MS calculated for C₂₅H₂₄F₃N₂O₄S (M+H⁺) 505.1, found 505.1.

Step B: The residue from Step A is dissolved in THF (3 mL) and 1 N LiOH (1 mL). The mixture is stirred at rt for 12 h then acidified with 1 N HCl (˜5 mL) and extracted with EtOAc (10 mL), dried (MgSO₄), filtered, evaporated and purified on reverse phase HPLC (H₂O/MeCN gradient) to afford the title compound B1 (32 mg, 32%): ¹H-NMR (400 MHz, CDCl₃) δ=8.01 (d, J=8.0 Hz, 2H), 7.82 (s, 1H), 7.71 (d, J=8.0 Hz, 2H), 7.11 (d, J=8.4 Hz, 1H), 6.82 (m, 2H), 4.89 (s, 2H), 4.60 (t, J=6.0 Hz, 2H), 3.06 (t, J=6.0 Hz, 1H), 1.61 (s, 6H). MS calculated for C₂₄H₂₁F₃N₂O₄S (M+H⁺) 490.1, found 490.1.

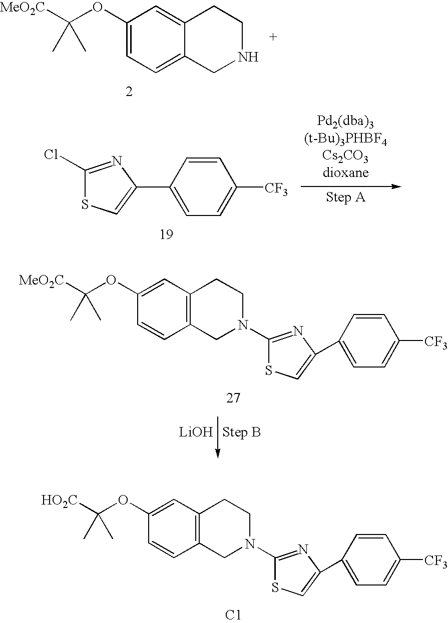

### Example C1

### 2-(2-(4-(4-(trifluoromethylphenyl)thiazol-2-yl)-1,2,3,4-tetrahydroisoquinolin-6-yloxy)-2-methylpronanoic acid

Step A: Methyl 2-(1,2,3,4-tetrahydroisoquinolin-6-yloxy)-2-methylpropanoate 2 (20 mg, 0.08 mmol), 2-chloro-4-(4-(trifluoromethyl)phenyl)thiazole 19 (21 mg, 0.08 mmol), (t-Bu)₃PHBF₄ (2.3 mg, 0.008 mmol), Pd₂(dba)₃ (3.6 mg, 0.004 mmol) and powdered cesium carbonate (52 mg, 0.16 mmol) are charged to a flame-dried vessel. Anhydrous 1,4-dioxane (0.4 mL) is added and the vessel is sealed, then flushed with argon and evacuated (3×) before being filled with argon and heated at 120° C. for 18 h to give crude methyl 2-(2-(4-(4-(trifluoromethylphenyl)thiazol-2-yl)-1,2,3,4-tetrahydroisoquinolin-6-yloxy)-2-methylpropanoate 27. MS calculated for C₂₄H₂₄F₃N₂O₃S (M+H) 477.1, found 477.1.

Step B: THF (1 mL) and 1 N LiOH (0.5 mL) are added directly to the reaction mixture from Step A and stirred at rt for 3 h. The mixture is then acidified with 1 N HCl (˜5 mL) and extracted with EtOAc (10 mL), dried (MgSO₄), filtered, evaporated and purified on reverse phase HPLC (H₂O/MeCN gradient) to afford the title compound C1 (24 mg, 64%): ¹H-NMR (400 MHz, CDCl₃) δ=7.79 (d, J=8.0 Hz, 2H), 7.58 (d, J=8.4 Hz, 2H), 7.19 (s, 1H), 6.95 (d, J=8.0 Hz, 1H), 6.71 (m, 2H), 4.63 (s, 2H), 3.71 (t, J=6.0 Hz, 2H), 2.87 (t, J=6.0 Hz, 2H), 1.50 (s, 6H). MS calculated for C₂₃H₂₂F₃N₂O₃S (M+H⁺) 462.1, found 462.1.

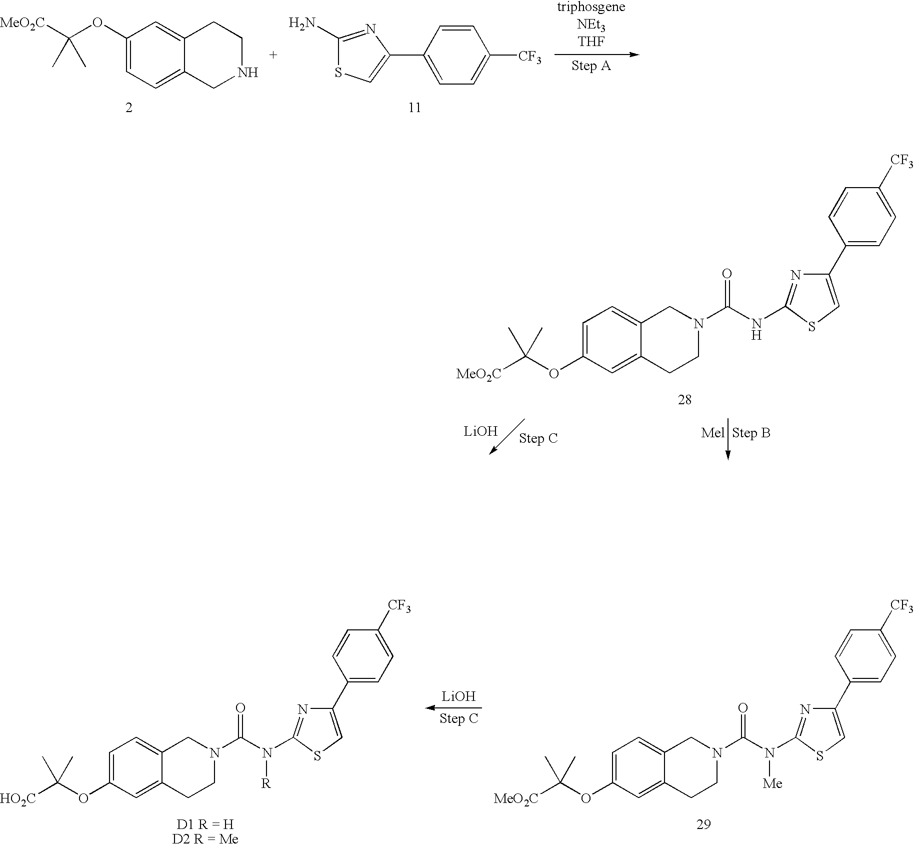

### Examples D1 & D2

### 2-(2-(4-(4-(Trifluoromethyl)phenyl)thiazol-2-ylcarbamoyl)-1,2,3,4-tetahydroisoquinolin-6-yloxyy-2-methylpropanoic acid D1 and 2-(2-(N-(4-(4-(trifluoromethyl)phenyl)thiazol-2-yl)-N-methylcarbamoyl)-1,2,3,4-tetrahydroisoquinolin-6-yloxy)-2-methylpropanoic acid D2

Step A: 4-(4-(trifluoromethyl)phenyl)thiazol-2-amine 11 (58 mg, 0.24 mmol) is dissolved in anhydrous THF (5 mL) and triethylamine (100 μL, 0.72 mmol) and cooled to 0° C. Triphosgene (24 mg, 0.08 mmol) is added and stirred at 0° C. for 5 min, then methyl 2-(1,2,3,4-tetrahydroisoquinolin-6-yloxy)-2-methylpropanoate 2 (60 mg, 0.24 mmol) is added and the mixture is stirred at rt for 3 h. The reaction mixture is diluted with water (10 mL) and extracted with EtOAc (10 mL), washed with brine (5 mL), dried (MgSO₄), filtered, evaporated and purified on reverse phase HPLC (H₂O/MeCN gradient) to afford methyl 2-(2-(4-(4-(trifluoromethyl)phenyl)thiazol-2-ylcarbamoyl)-1,2,3,4-tetrahydroisoquinolin-6-yloxy)-2-methylpropanoate 28 (34 mg, 27%): ¹H-NMR (400 MHz, CDCl₃) δ=7.88 (d, J=8.0 Hz, 2H), 7.66 (d, J=8.4 Hz, 2H), 7.16 (s, 1H), 6.99 (d, J=8.0 Hz, 1H), 6.67 (m, 2H), 4.62 (s, 2H), 3.78 (s, 3H), 3.75 (t, J=6.0 Hz, 2H), 3.59 (m, 2H), 2.86 (t, J=6.0 Hz, 2H), 1.59 (s, 6H). MS calculated for C₂₅H₂₅F₃N₃O₄S (M+H⁺) 520.1, found 520.1.

Step B: (for D2 only) Methyl 2-(2-(4-(4-(trifluoromethyl)phenyl)thiazol-2-ylcarbamoyl)-1,2,3,4-tetrahydroisoquinolin-6-yloxy)-2-methylpropanoate 28 (20 mg, 0.038 mmol) is dissolved in dry acetonitrile (5 mL). Powdered cesium carbonate (44 mg, 0.13 mmol) and iodomethane (8 μL, 0.11 mmol) are added and the mixture is stirred at rt for 2 h. The reaction mixture is diluted with water (10 mL) and extracted with EtOAc (10 mL), washed with brine (5 mL), dried (MgSO₄), filtered and evaporated to give crude methyl 2-(2-(N-(4-(4-(trifluoromethyl)phenyl)thiazol-2-yl)-N-methylcarbamoyl)-1,2,3,4-tetrahydroisoquinolin-6-yloxy)-2-methylpropanoate 29, which is used without further purification in Step C.

Step C: Intermediate 28 (D1) or 29 (D2) is dissolved in THF (3 mL) then 1 N LiOH (1.0 mL) is added stirred at rt for 4 h. The mixture is acidified with 1 N HCl (˜5 mL) and extracted with EtOAc (10 mL), dried (MgSO₄), filtered, evaporated and purified on reverse phase HPLC (H₂O/MeCN gradient) to afford the title compounds D1 (18 mg, 92%). ¹H-NMR (400 MHz, CDCl₃) δ=7.77 (d, J=8.0 Hz, 2H), 7.68 (d, J=8.4 Hz, 2H), 7.07 (s, 1H), 6.90 (d, J=8.0 Hz, 1H), 6.79 (dd, J=2.4, 8.0 Hz, 1H), 6.73 (s, 1H), 4.46 (s, 2H), 3.61 (br s, 2H), 2.68 (t, J=5.6 Hz, 2H), 1.69 (s, 6H). MS calculated for C₂₄H₂₃F₃N₃O₄S (M+H⁺) 506.1, found 506.1 and D2 (8 mg, 40%): ¹H-NMR (400 MHz, CDCl₃) δ=7.96 (d, J=8.0 Hz, 2H), 7.64 (d, J=8.0 Hz, 2H), 7.18 (s, 1H), 7.01 (d, J=8.4 Hz, 1H), 6.78 (m, 2H), 4.55 (s, 2H), 3.66 (t, J=6.0 Hz, 2H), 3.64 (s, 3H), 2.96 (t, J=6.0 Hz, 2H), 1.60 (s, 6H). MS calculated for C₂₅H₂₅F₃N₃O₄S (M+H⁺) 520.1, found 520.1.

By repeating the procedures described in the above examples, using appropriate starting materials, the following compounds of Formula 1, as identified in Table 1, are obtained.

TABLE 1

| Compound | Compound | Physical Data |
|---|---|---|
| Number | Structure | ¹H NMR and/or MS (m/z) |
| A2 | 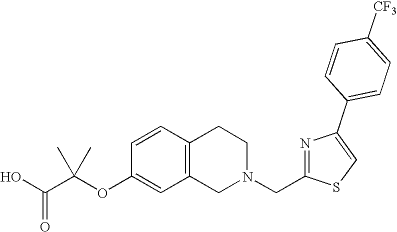 | ¹H-NMR (400 MHz, CDCl₃) δ =7.98 (d, J = 8.0 Hz, 2H), 7.74 (s,1H), 7.68 (d, J = 8.0 Hz, 2H), 7.06(d, J = 8.8 Hz, 1H), 6.84 (dd, J =2.4, 8.4 Hz, 1H), 6.66 (d, J = 2.4Hz, 1H), 4.74 (s, 2H), 4.44 (s,2H), 3.57 (br s, 2H), 3.14 (t, J =6.4 Hz, 2H), 1.57 (s, 6H). MScalculated for C₂₄H₂₄F₃N₂O₃S(M + H⁺) 477.2, found 477.1. |
| A3 | 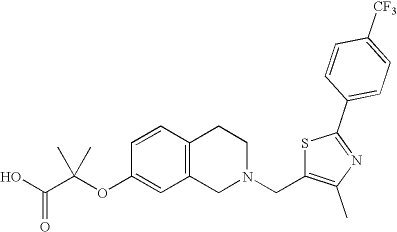 | ¹H-NMR (400 MHz, CDCl₃) δ =8.00 (d, J = 8.4 Hz, 2H), 7.68 (d, J =8.4 Hz, 2H), 7.01 (d, J = 8.4 Hz,1H), 6.81 (dd, J = 2.4, 8.4 Hz,1H), 6.61 (d, J = 2.4 Hz, 1H), 4.57(s, 2H), 4.28 (s, 2H), 3.51 (br s,2H), 3.09 (s, 2H), 2.44 (s, 3H),1.57 (s, 6H). MS calculated forC₂₅H₂₆F₃N₂O₃S (M + H⁺) 491.2,found 491.1. |
| A4 | 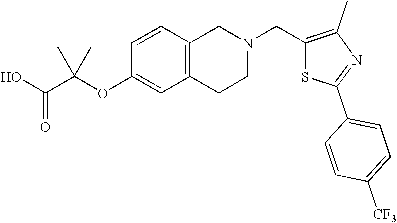 | MS calculated for C₂₅H₂₆F₃N₂O₃S(M + H⁺) 491.2, found 491.1. |
| A5 | 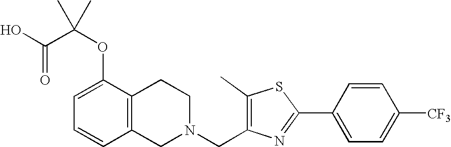 | ¹H-NMR (400 MHz, CDCl₃) δ =7.96 (d, J = 8.0 Hz, 2H), 7.62 (d, J =8.0 Hz, 2H), 7.02 (t, J = 8.0 Hz,1H), 6.65 (d, J = 8.0 Hz, 1H), 6.58(d, J = 8.0 Hz, 1H), 4.48 (s, 2H),4.21 (s, 2H), 3.46 (s, 2H), 3.05 (s,2H), 2.33 (s, 3H), 1.58 (s, 6H).MS calculated for C₂₅H₂₆F₃N₂O₃S(M + H⁺) 491.2, found 491.2. |
| A6 | 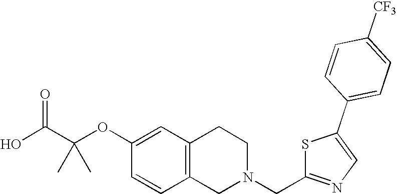 | ¹H-NMR (400 MHz, CDCl₃) δ =8.00 (s, 1H), 7.60 (s, 4H), 6.79 (d,J = 8.4 Hz, 1H), 6.63 (m, 2H),4.70 (s, 2H), 4.35 (s, 2H), 3.46 (s,2H), 3.05 (s, 2H), 1.51 (s, 6H). MS calculated for C₂₄H₂₄F₃N₂O₃S (M + H⁺)477.1, found 477.1. |
| A7 | 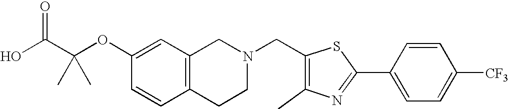 | ¹H-NMR (400 MHz, CDCl₃) δ =7.95 (d, J = 8.0 Hz, 2H), 7.64 (m,3H), 7.07 (m, 2H), 6.80 (br s, 2H),4.88 (s, 2H), 3.57 (t, J = 6.4 Hz,2H), 2.92 (t, J = 6.4 Hz, 2H), 2.53(s, 3H), 1.61 (s, 6H). MScalculated for C₂₄H₂₄F₃N₂O₃S(M + H⁺) 505.1, found 505.1. |
| A8 | 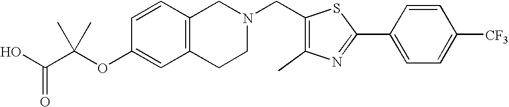 | ¹H-NMR (400 MHz, CDCl₃) δ =7.96 (m, 3H), 7.67 (d, J = 8.4 Hz,2H), 6.74 (d, J = 8.8 Hz, 1H), 6.72(d, J = 2.0 Hz, 1H), 4.90 (s, 2H),3.60 (t, J = 6.4 Hz, 2H), 2.95 (t, J =6.4 Hz, 2H), 2.55 (s, 3H), 1.67(s, 6H). MS calculated forC₂₄H₂₄F₃N₂O₃S (M + H⁺) 505.1,found 505.2. |
| B2 | 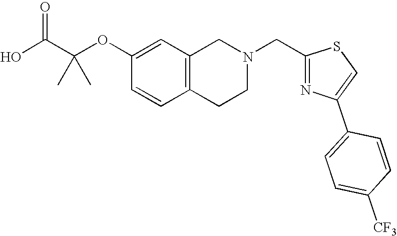 | ¹H-NMR (400 MHz, CDCl₃) δ =8.01 (d, J = 8.4 Hz, 2H), 7.82 (d, J =3.2 Hz, 1H), 7.72 (t, J = 7.6 Hz,2H), 7.10 (d, J = 8.0 Hz, 1H), 6.80(m, 2H), 4.89 (s, 2H), 4.60 (t, J =6.0 Hz, 2H), 3.04 (t, J = 5.6 Hz,2H), 1.61 (s, 6H). MS calculatedfor C₂₄H₂₂F₃N₂O₄S (M + H⁺) 491.1,found 491.1. |
| B3 | 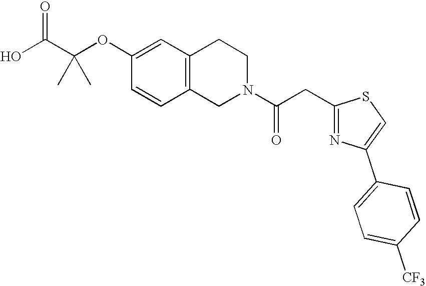 | ¹H-NMR (400 MHz, CDCl₃) δ =7.96 (d, J = 8.0 Hz, 2H), 7.67 (d, J =8.0 Hz, 2H), 7.56 (s, 1H), 7.04(d, J = 8.4 Hz, 1H), 6.79 (d, J =2.4 Hz, 1H), 6.75 (m, 1H), 4.73 (s,2H), 4.30 (s, 2H), 3.86 (t, J = 6.0Hz, 2H), 2.84 (t, J = 5.6 Hz, 2H),1.59 (s, 6H). MS calculated forC₂₅H₂₄F₃N₂O₄S (M + H⁺) 505.1,found 505.2. |
| D3 | 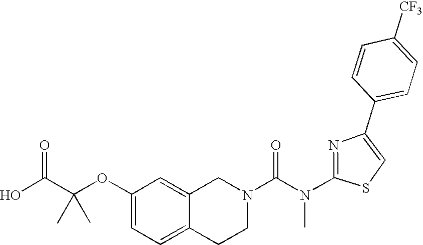 | ¹H-NMR (400 MHz, CDCl₃) δ =7.96 (d, J = 8.0 Hz, 2H), 7.64 (d, J =8.0 Hz, 2H), 7.18 (s, 1H), 7.09(d, J = 8.4 Hz, 1H), 6.80 (dd, J =2.4, 8.4 Hz, 1H), 6.70 (d, J = 2.4Hz, 1H), 4.55 (s, 2H), 3.68 (t, J =6.0 Hz, 2H), 3.64 (s, 3H), 2.95 (t, J =6.0 Hz, 2H), 1.58 (s, 6H). MScalculated for C₂₅H₂₅F₃N₃O₄S(M + H⁺) 520.1, found 520.1. |
| D4 | 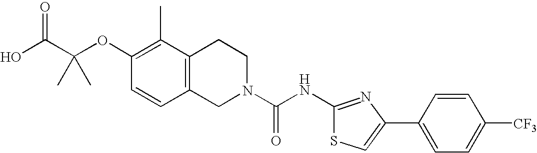 | ¹H-NMR (400 MHz, DMSO-d6) δ =8.12 (d, J = 8.0 Hz, 2H), 7.78 (d,J = 8.4 Hz, 2H), 7.72 (s, 1H), 6.92(d, J = 8.4 Hz, 1H), 6.65 (d, J =8.4 Hz, 1H), 4.64 (s, 2H), 3.81 (m,2H), 2.74 (t, J = 6.0 Hz, 2H), 2.09(s, 3H), 1.48 (s, 6H). MScalculated for C₂₅H₂₅F₃N₃O₄S(M + H⁺) 520.1, found 520.1. |
| D5 | 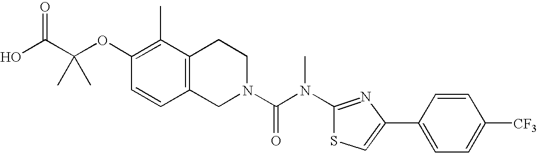 | ¹H-NMR (400 MHz, CDCl₃) δ =7.96 (d, J = 8.4 Hz, 2H), 7.64 (d, J =8.4 Hz, 2H), 7.17 (s, 1H), 6.84(d, J = 8.4 Hz, 1H), 6.73 (d, J =8.4 Hz, 1H), 4.53 (s, 2H), 3.69 (t, J =6.0 Hz, 2H), 3.65 (s, 3H), 2.88(t, J = 6.0 Hz, 2H), 2.16 (s, 3H),1.60 (s, 6H). MS calculated forC₂₆H₂₇F₃N₃O₄S (M + H⁺) 534.2,found 534.1. |

### Transcriptional Assay

Transfection assays are used to assess the ability of compounds of the invention to modulate the transcriptional activity of the PPARs. Briefly, expression vectors for chimeric proteins containing the DNA binding domain of yeast GAL4 fused to the ligand-binding domain (LBD) of either PPARδ, PPARα or PPARγ are introduced via transient transfection into mammalian cells, together with a reporter plasmid where the luciferase gene is under the control of a GAL4 binding site. Upon exposure to a PPAR modulator, PPAR transcriptional activity varies, and this can be monitored by changes in luciferase levels. If transfected cells are exposed to a PPAR agonist, PPAR-dependent transcriptional activity increases and luciferase levels rise.

293T human embryonic kidney cells (8×10⁶) are seeded in a 175 cm² flask a day prior to the start of the experiment in 10% FBS, 1% Penicillin/Streptomycin/Fungizome, DMEM Media. The cells are harvested by washing with PBS (30 ml) and then dissociating using trypsin (0.05%; 3 ml). The trypsin is inactivated by the addition of assay media (DMEM, CA-dextran fetal bovine serum (5%). The cells are spun down and resuspended to 170,000 cells/ml. A Transfection mixture of GAL4-PPAR LBD expression plasmid (1 μg), UAS-luciferase reporter plasmid (1 μg), Fugene (3:1 ratio; 6 μL) and serum-free media (200 μL) was prepared and incubated for 15-40 minutes at room temperature. Transfection mixtures are added to the cells to give 0.16M cells/mL, and cells (50 μl/well) are then plated into 384 white, solid-bottom, TC-treated plates. The cells are further incubated at 37° C., 5.0% CO₂ for 5-7 hours. A 12-point series of dilutions (3 fold serial dilutions) are prepared for each test compound in DMSO with a starting compound concentration of 10 μM. Test compound (500 nl) is added to each well of cells in the assay plate and the cells are incubated at 37° C., 5.0% CO₂ for 18-24 hours. The cell lysis/luciferase assay buffer, Bright-Glom (25%; 25 μl; Promega), is added to each well. After a further incubation for 5 minutes at room temperature, the luciferase activity is measured.

Raw luminescence values are normalized by dividing them by the value of the DMSO control present on each plate. Normalized data is analyzed and dose-response curves are fitted using Prizm graph fitting program. EC50 is defined as the concentration at which the compound elicits a response that is half way between the maximum and minimum values. Relative efficacy (or percent efficacy) is calculated by comparison of the response elicited by the compound with the maximum value obtained for a reference PPAR modulator.

Compounds of Formula I, in free form or in pharmaceutically acceptable salt form, exhibit valuable pharmacological properties, for example, as indicated by the in vitro tests described in this application. Compounds of the invention preferably have an EC50 for PPARδ and/or PPARα and/or PPARγ, of less than 5 μM more preferably less than 1 μM more preferably less than 500 nm, more preferably less than 100 nM. Compounds of the invention preferably have an EC50 for PPARδ that is less than or equal to PPARα which in turn has an EC50 that is at least 10-fold less than PPARγ.

It is understood that the examples and embodiments described herein are for illustrative purposes only and that various modifications or changes in light thereof will be suggested to persons skilled in the art and are to be included within the spirit and purview of this application and scoped of the appended claims. All publications, patents, and patent applications cited herein are hereby incorporated by reference for all purposes.

We claim:

1. A compound of Formula I: 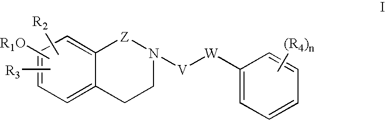
in which:
n is selected from 0, 1 and 2;
R₁ is —CR₁₁R₁₂XCO₂R₁₃; wherein X is selected from a bond and C₁₋₄alkylene; and R₁₁ and R₁₂ are independently selected from hydrogen, C₁₋₄alkyl and C₁₋₄alkoxy; or R₁₁ and R₁₂ together with the carbon atom to which R₁₁ and R₁₂ are attached form C₃₋₁₂cycloalkyl; and R₁₃ is selected from hydrogen and C₁₋₆alkyl;
R₂ and R₃ are independently selected from hydrogen and C₁₋₆alkyl;
V is selected from a bond, C₁₋₄alkylene, —C(O)NR₈— and —X₁C(O)X₂—; wherein X₁ and X₂ are independently selected from a bond and C₁₋₄alkylene; R₈ is selected from hydrogen and C₁₋₆alkyl;
W is a divalent radical selected from (a) and (b): 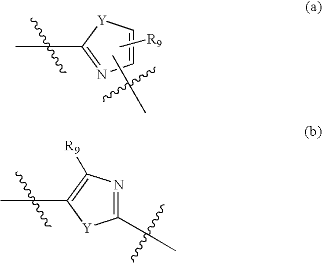
wherein Y is selected from O and S; and R₉ is selected from hydrogen and C₁₋₆alkyl;
Z is selected from —CH₂— and —C(O)—;
R₄ is selected from hydrogen, halo, C₁₋₆alkyl, halo-substituted-C₁₋₆alkyl, C₁₋₆alkoxy and halo-substituted-C₁₋₆alkoxy; and the pharmaceutically acceptable salts, hydrates, solvates and isomers thereof.

2. The compound of claim 1 in which:
n is selected from 0 and 1;
R₁ is —OCR₁₁R₁₂XCO₂R₁₃; wherein X is selected from a bond and C₁₋₄alkylene; and
R₁₁ and R₁₂ are independently selected from hydrogen, C₁₋₄alkyl and C₁₋₄alkoxy; or R₁₁ and R₁₂ together with the carbon atom to which R₁₁ and R₁₂ are attached form C₃₋₁₂cycloalkyl; and R₁₃ is selected from hydrogen and C₁₋₆alkyl;
R₂ and R₃ are independently selected from hydrogen and C₁₋₆alkyl;
V is selected from a bond, C₁₋₄alkylene, —C(O)NR₈— and —X₁C(O)X₂—; wherein X₁ and X₂ are independently selected from a bond and C₁₋₄alkylene; R₈ is selected from hydrogen and C₁₋₆alkyl;
W is a divalent radical selected from (a) and (b): 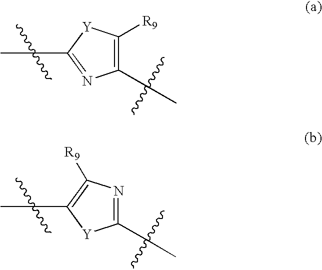
wherein Y is S; and R₉ is selected from hydrogen and C₁₋₄alkyl;
Z is selected from —CH₂ and —C(O)—;
R₄ is selected from halo, C₁₋₆alkyl and halo-substituted-C₁₋₆alkyl.

3. The compound of claim 2 in which: R₁ is selected from —CH₂CO₂H, —CH₂)₂CO₂H, —OC(CH₂)₂CO₂H and —OCH₂CO₂H; R₂ and R₃ are independently selected from hydrogen, methyl and methoxy; and R₄ is trifluoromethyl.

4. The compound of claim 3 in which: V is selected from a bond, —C(O)—, —C(O)NH—, —C(O)N(CH₃)—, —CH₂— and —C(O)CH₂—.

5. The compound of claim selected from: 2-(2-(4-(4-(Trifluoromethyl)phenyl)-thiazol-2-ylcarbamoyl)-1,2,3,4-tetrahydroisoquinolin-6-yloxy)-2-methylpropanoic acid; 2-(2-(N-(4-(4-(trifluoromethyl)phenyl)thiazol-2-yl)-N-methyl-carbamoyl)-1,2,3,4-tetrahydro-isoquinolin-6-yloxy)-2-methylpropanoic acid; 2-(2-(4-(4-(trifluoromethyl)phenyl)thiazol-2-yl)-1,2,3,4-tetrahydroisoquinolin-6-yloxy)-2-methylpropanoic acid; 2-(2-((4-(4-(trifluoromethyl)phenyl)thiazol-2-yl)(oxo)methyl)-1,2,3,4-tetrahydroisoquinolin-6-yloxy)-2-methylpropanoic acid; 2-(2-((4-(4-(trifluoromethyl)phenyl)thiazol-2-yl)methyl)-1,2,3,4-tetrahydroisoquinolin-6-yloxy)-2-methylpropanoic acid; 2-Methyl-2-{2-[4-(4-trifluoromethyl-phenyl)-thiazol-2-ylmethyl]-1,2,3,4-tetrahydro-isoquinolin-7-yloxy}-propionic acid; 2-Methyl-2-{2-[4-methyl-2-(4-trifluoromethyl-phenyl)-thiazol-5-ylmethyl]-1,2,3,4-tetrahydro-isoquinolin-7-yloxy}-propionic acid; 2-Methyl-2-{2-[4-methyl-2-(4-trifluoromethyl-phenyl)-thiazol-5-ylmethyl]-1,2,3,4-tetrahydro-isoquinolin-6-yloxy}-propionic acid; 2-Methyl-2-{2-[5-methyl-2-(4-trifluoromethyl-phenyl)-thiazol-4-ylmethyl]-1,2,3,4-tetrahydro-isoquinolin-5-yloxy}-propionic acid; 2-Methyl-2-{2-[5-(4-trifluoromethyl-phenyl)-thiazol-2-ylmethyl]-1,2,3,4-tetrahydro-isoquinolin-6-yloxy}-propionic acid; 2-Methyl-2-{2-[4-methyl-2-(4-trifluoromethyl-phenyl)-thiazol-5-ylmethyl]-1-oxo-1,2,3,4-tetrahydro-isoquinolin-7-yloxy}-propionic acid; 2-Methyl-2-{2-[4-methyl-2-(4-trifluoromethyl-phenyl)-thiazol-5-ylmethyl]-1-oxo-1,2,3,4-tetrahydro-isoquinolin-6-yloxy}-propionic acid; 2-Methyl-2-{2-[4-(4-trifluoromethyl-phenyl)-thiazole-2-carbonyl]-1,2,3,4-tetrahydro-isoquinolin-7-yloxy}-propionic acid; 2-Methyl-2-(2-{2-[4-(4-trifluoromethyl-phenyl)-thiazol-2-yl]-acetyl}-1,2,3,4-tetrahydro-isoquinolin-6-yloxy)propionic acid; 2-Methyl-2-(2-n{methyl-[4-(4-trifluoromethyl-phenyl)-thiazol-2-yl]-carbamoyl}-1,2,3,4-tetrahydro-isoquinolin-7-yloxy)-propionic acid; 2-Methyl-2-{5-methyl-2-[4-(4-trifluoromethyl-phenyl)-thiazol-2-ylcarbamoyl]-1,2,3,4-tetrahydro-isoquinolin-6-yloxy}-propionic acid; and 2-Methyl-2-(5-methyl-2-{methyl-[4-(4-trifluoromethyl-phenyl)thiazol-2-yl]-carbamoyl}-1,2,3,4-tetrahydro-isoquinolin-6-yloxy)-propionic acid.

6. A method for treating a disease or disorder in an animal in which modulation of PPAR activity can prevent, inhibit or ameliorate the pathology and/or symptomology of the disease, which method comprises administering to the animal a therapeutically effective amount of a compound of claim 1.

7. The method of claim 6 in which the PPAR activity is at least one PPAR selected from PPARα, PPARδ and PPARγ.

8. The method of claim 7 in which the PPAR activity is both PPARα and PPARδ.

9. The method of claim 6 in which the disease or disorder is selected from the treatment of prophylaxis, dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, cachexia, inflammation, arthritis, cancer, anorexia, anorexia nervosa, bulimia, Alzheimer's disease, skin disorders, respiratory diseases, ophthalmic disorders, irritable bowel diseases, ulcerative colitis, Crohn's disease, type-1 diabetes, type-2 diabetes and Syndrome X.

10. The method of claim 6 in which the disease or disorder is selected from HIV wasting syndrome, long term critical illness, decreased muscle mass and/or muscle strength, decreased lean body mass, maintenance of muscle strength and function in the elderly, diminished muscle endurance and muscle function, and frailty in the elderly.

11. The use of a compound according to any of claims 1 to 5 in the manufacture of a medicament for treating a disease in an animal in which PPAR activity contributes to the pathology and/or symptomology of the disease.

12. The use of claim 11 in which the PPAR activity is at least one PPAR selected from PPARα, PPARδ and PPARδ.

13. The use of claim 12 in which the PPAR activity is both PPARα and PPARδ.

14. A pharmaceutical composition comprising a therapeutically effective amount of a compound of any of claim 1 to 5 in combination with one or more pharmaceutically acceptable excipients.

15. A pharmaceutical combination, especially a pharmaceutical composition, comprising: 1) a compound of any of claims 1 to 5 or a pharmaceutical acceptable salt thereof; and 2) at least one active ingredient selected from:
a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; dipeptidyl peptidase IV inhibitors such as DPP728, vildagliptin, MK-0431, saxagliptin, GSK23A; an AGE breaker; a thiazolidone derivative (glitazone) such as pioglitazone, rosiglitazone, or (R)-1-{4-[5-methyl-2-(4-trifluoromethylphenyl)-oxazol-4-ylmethoxy]-benzenesulfonyl}-2,3-dihydro-1H-indole-2-carboxylic acid, a non-glitazone type PPARγ agonist e.g. GI-262570;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;
d) anti-hypertensive agents, e.g., loop diuretics such as ethacrynic acid, furosemide and torsemide; diuretics such as thiazide derivatives, chlorithiazide, hydrochlorothiazide, amiloride, angiotensin converting enzyme (ACE) inhibitors such as benazepril, captopril, enalapril, fosinopril, lisinopril, moexipril, perindopril, quinapril, ramipril and trandolapril; inhibitors of the Na-K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;
e) a HDL increasing compound;
f) a cholesterol absorption modulator such as Zetia® and KT6-971;
g) Apo-A1 analogues and mimetics;
h) thrombin inhibitors such as Ximelagatran;
i) aldosterone inhibitors such as anastrazole, fadrazole, eplerenone;
j) Inhibitors of platelet aggregation such as aspirin, clopidogrel bisulfate;
k) estrogen, testosterone, a selective estrogen receptor modulator, a selective androgen receptor modulator;
l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib or 4-Methyl-N-[3-(4-methyl-imidazol-1-yl)-5-trifluoromethyl-phenyl]-3-(4-pyridin-3-ylpyrimidine-2-ylamino)-benzamide; and
m) an agent interacting with a 5-HT₃ receptor and/or an agent interacting with 5-HT₄ receptor such as tegaserod, tegaserod hydrogen maleate, cisapride, cilansetron;
or, in each case a pharmaceutically acceptable salt thereof; and optionally a pharmaceutically acceptable carrier.

16. A pharmaceutical composition according to claim 14 or a combination according to claim 15, for the treatment or prevention of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.

17. A compound according to any of claims 1 to 5, or a pharmaceutical composition according to claim 10 or a combination according to claim 11, for use as a medicament.

18. Use of a compound according to any of claims 1 to 5, or a pharmaceutical composition according to claim 14 or a combination according to claim 15, for the manufacture of a medicament for the treatment or prevention of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's, disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.
